(19) United States
(12) Patent Application Publication
(10) Pub. No.: US 2011/0046736 A1
(43) Pub. Date: Feb. 24, 2011

(54) INTERVERTEBRAL STABILIZATION ASSEMBLY FOR ARTHRODESIS, COMPRISING AN IMPACTION CAGE BODY, AND AN ANCILLARY DEVICE FOR IMPLANTING SAME

(76) Inventor: Henry Graf, Lyon (FR)

Correspondence Address: DOWELL & DOWELL P.C., 103 Oronoco St., Suite 220, Alexandria, VA, 22314, US

(21) Appl. No.: 12/812,637

(22) PCT Filed: Jan. 14, 2009
(86) PCT No.: PCT/FR2009/050048

§ 371 Date: Nov. 10, 2010

(30) Foreign Application Priority Data
Jan. 15, 2008 (FR) 08 50220
Apr. 3, 2008 (FR) 08 52208
Sep. 30, 2008 (FR) 08 56560

(51) Int. Cl. A61F 2/44

(52) U.S. Cl. 623/17.11

(57) ABSTRACT

This stabilization assembly comprises a solid cage body (1) designed to be introduced by impaction into the intervertebral space (E), and a nose (36) for introducing the cage body into the intervertebral space, this nose being integrally connected or being able to be integrally connected to this cage body in an eccentric position in such a way that, when this nose is introduced into the intervertebral space, the rotation of this nose induces an opening of this intervertebral space and, simultaneously, a displacement of the cage body in the manner of a cam.

The present invention relates to an intervertebral stabilization assembly comprising an impaction cage body, and to an ancillary device for implanting this stabilization assembly.

The invention falls within the field of arthrodesis, namely the fusing together of the bone of at least two adjacent vertebrae. It will be recalled that the aim of arthrodesis is to allow only micromovements between the vertebrae, and to deaden vibration. These micromovements amongst other things allow the patient, once he has become ambulant again following the operation, to regain his balance as best he can before the bone graft takes.

Typically, an extra-discal assembly for arthrodesis, within the meaning of the invention, allows a range of movement between two vertebrae which is at best equal to about 10% of the natural physiological range. In other words, if there is a maximum natural range of 10° in rotation between two given vertebrae, the stabilization assembly according to the invention is at best able to allow a travel of 1° between these two vertebrae. This authorized range of movement is established about a chosen position, determined by the surgeon at the time of the operation, notably according to the pathology. It will also be noted that this chosen position, while it does of course fall within the authorized range of movement, does not necessarily lie in the middle thereof.

Usually, an intervertebral cage is intended to replace all or part of a disk when the latter has been destroyed by surgery or disease. The main function of such a cage is to restore the discal height, namely the spacing between two facing vertebral bodies. It also performs an anterior stabilization function, allowing bone fusion to be created, in the case of arthrodesis.

The invention is aimed more specifically at a stabilization assembly which comprises a cage body that can be introduced by impaction into the intervertebral space. The conventional implantation method associated with this type of cage first of all makes use of vertebral dilating bougies of increasing size, the dimensions of which typically vary from 8 to 13 mm.

The intervertebral space is dilated by rotating by a quarter of a turn so that the bougie finds itself edge-on, separating the vertebrae from one another by a distance corresponding to the width of this bougie. Once the volume necessary for introducing the cage has been created, this cage is impacted into the intervertebral space.

Conventional cages have disadvantages associated in particular with the way in which they are implanted. Specifically, these methods prove to be somewhat inconvenient to implement, or even dangerous to the patient, inasmuch as the nerve root is manipulated fairly roughly. Furthermore, the taller the cage, the more difficult this impaction maneuver is to perform.

The invention aims to address these various disadvantages. In particular, it aims to provide an intervertebral stabilization assembly the structure of which allows a cage body to be implanted into the intervertebral space in a way that is simple and less dangerous to the patient.

To this end, the subject of the invention is an intervertebral stabilization assembly for arthrodesis, comprising a cage body, intended to be introduced by impaction into the intervertebral space, this assembly further comprising a nose for introducing the cage body into the intervertebral space, this nose being secured to or able to be secured to this cage body, in an eccentric position, so that when this nose is introduced into the intervertebral space, the rotating of this nose causes this intervertebral space to open up and, at the same time, causes the cage body to move in the manner of a cam.

A further subject of the invention is a method for implanting the above assembly, comprising the following steps:
the nose is introduced into the intervertebral space via its smallest dimension, so as to give this intervertebral space a first height, leaving the cage body outside this intervertebral space;
the nose is turned about its main axis, still leaving the cage body outside the intervertebral space, so as to increase the height of this intervertebral space; and
the cage body is introduced axially into the intervertebral space thus made taller.

According to other features:
the nose is introduced to the side of the intervertebral space, with reference to the sagittal median axis, arranging that part of the cage body that is on the opposite side to the nose in such a way that it projects toward the center of the spinal column when the nose is rotated, so as to move the nerve root aside;
the nose is turned in such a way as to increase the height of the intervertebral space, then the cage body is inserted axially;
the nose is turned at the same time as the cage body is inserted axially;
the nose is immobilized with respect to the cage body before this nose is inserted into the intervertebral space and, once this cage has been positioned in the intervertebral space, the nose is detached from the cage body;
the nose is detached by unscrewing the threaded rod of the grasping member from the tapped mount of the nose;
the cage body is secured with respect to the implanting member so that this grasping member forms the insertion nose, then this grasping member is detached after the cage has been positioned;
in order to detach the member from the cage body, this cage body is pushed back by means of the piston so as to cause the branches to emerge from the grooves;
the first insert is positioned in the envelope of the cage body, so as to form the insertion nose and then, after the cage body has been inserted into the intervertebral space, this first insert is removed from the interior volume of the envelope and at least one additional insert is possibly introduced into this envelope.

The invention will be described hereinafter with reference to the attached drawings, given solely by way of nonlimiting examples, in which:

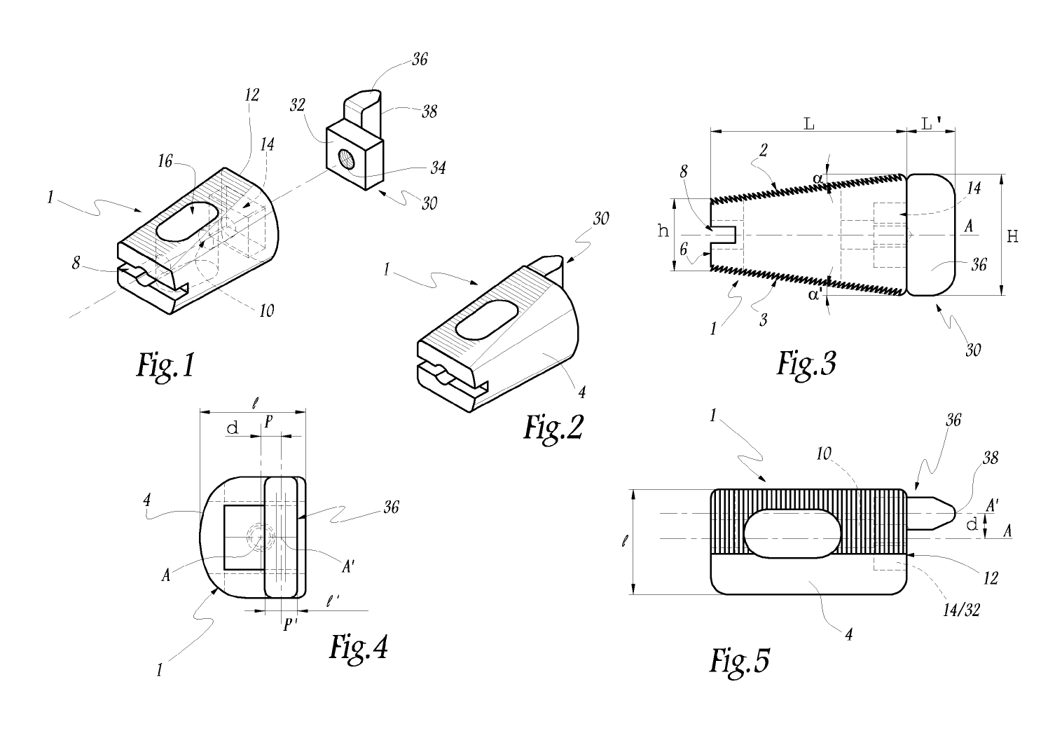

FIGS. 1 and 2 and 3 to 5 are respectively perspective, side, front and top views illustrating a stabilization assembly according to the invention;

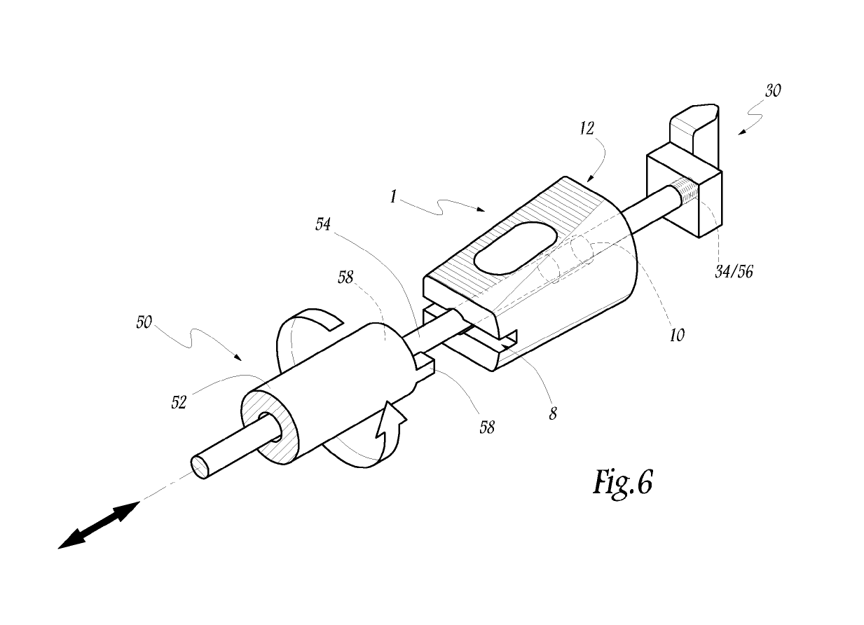

FIG. 6 is a perspective view illustrating an ancillary for implanting the stabilization assembly of FIGS. 1 to 5;

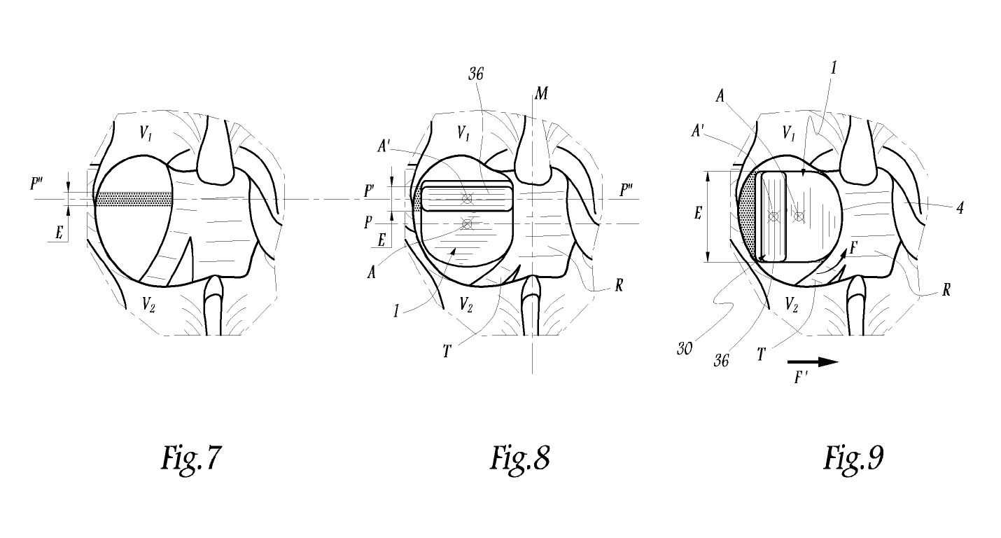

FIGS. 7 to 9 are rear views illustrating the placement of the stabilization assembly of FIGS. 1 to 5 using the ancillary of FIG. 6;

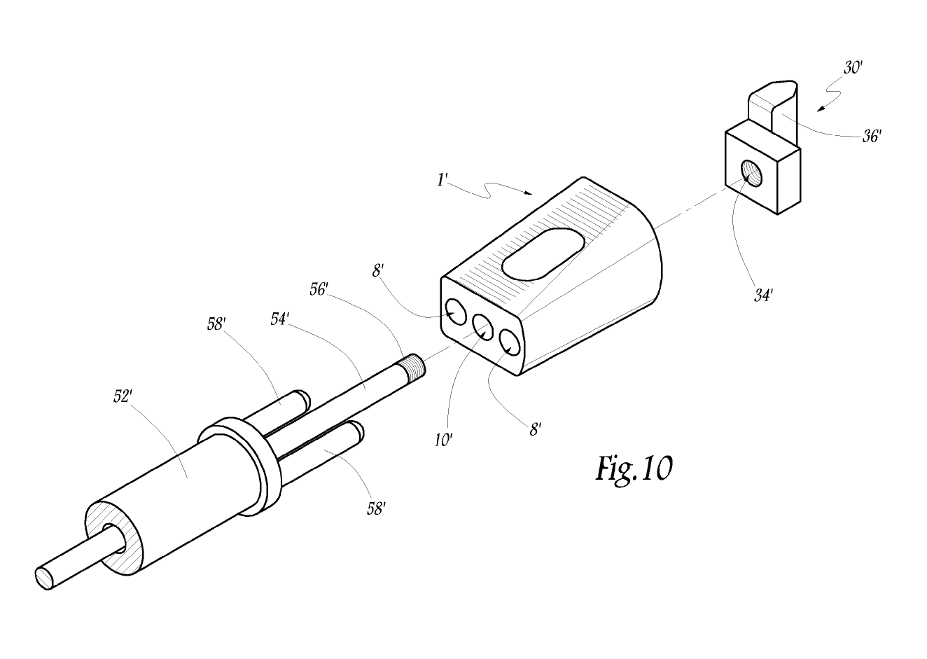

FIG. 10 is a perspective view similar to FIG. 6, illustrating a first alternative form of the invention,

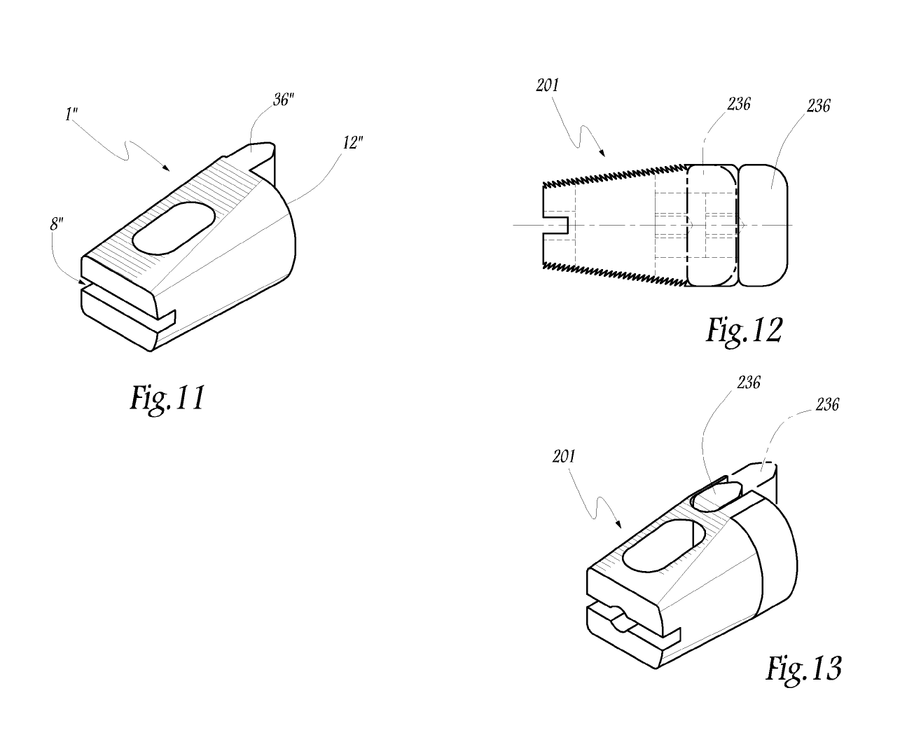

FIG. 11 is a perspective view similar to FIG. 2, illustrating another alternative form of the invention;

FIGS. 12 to 13 are respectively side and perspective views illustrating another alternative form of the invention;

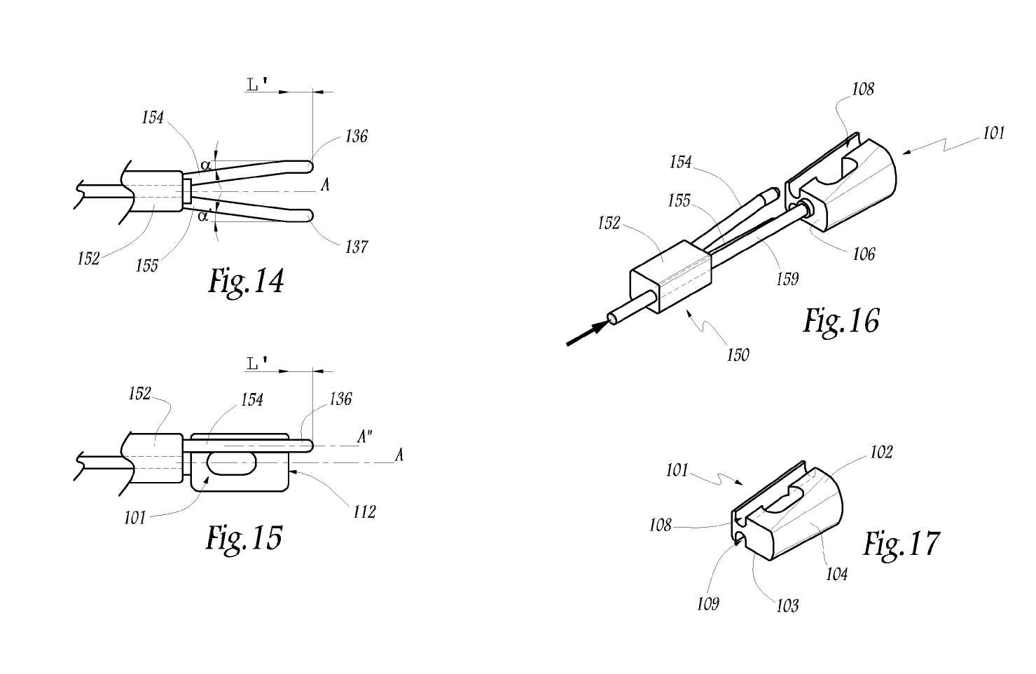

FIGS. 14 to 17 are respectively side and perspective views illustrating yet another alternative form of the invention;

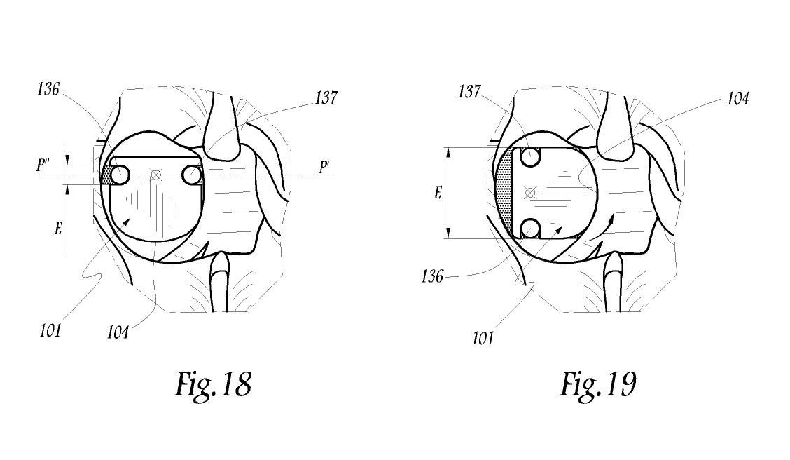

FIGS. 18 and 19 are rear views similar to FIGS. 6 to 8, illustrating the placement of the stabilization assembly of FIGS. 14 to 17;

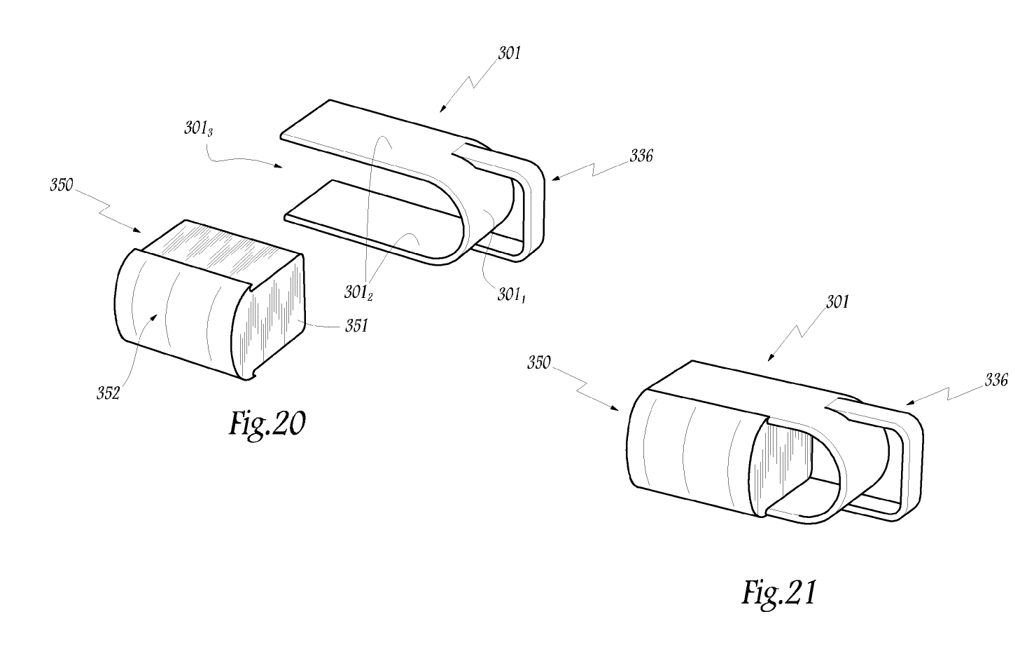

FIGS. 20 and 21 are perspective views illustrating the implementation of another alternative form of the invention;

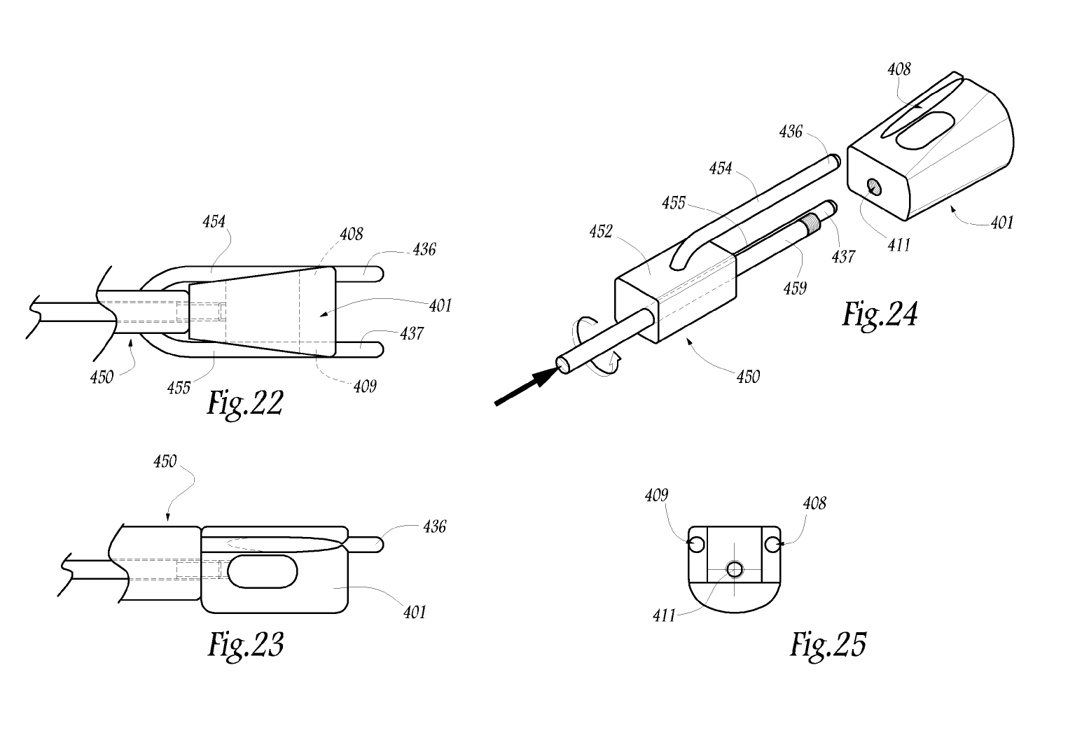

FIGS. 22 to 24 are respectively top, side and perspective views illustrating an additional alternative form of the invention;

FIG. 25 is a rear view illustrating a cage body involved in the embodiment of FIGS. 22 to 24;

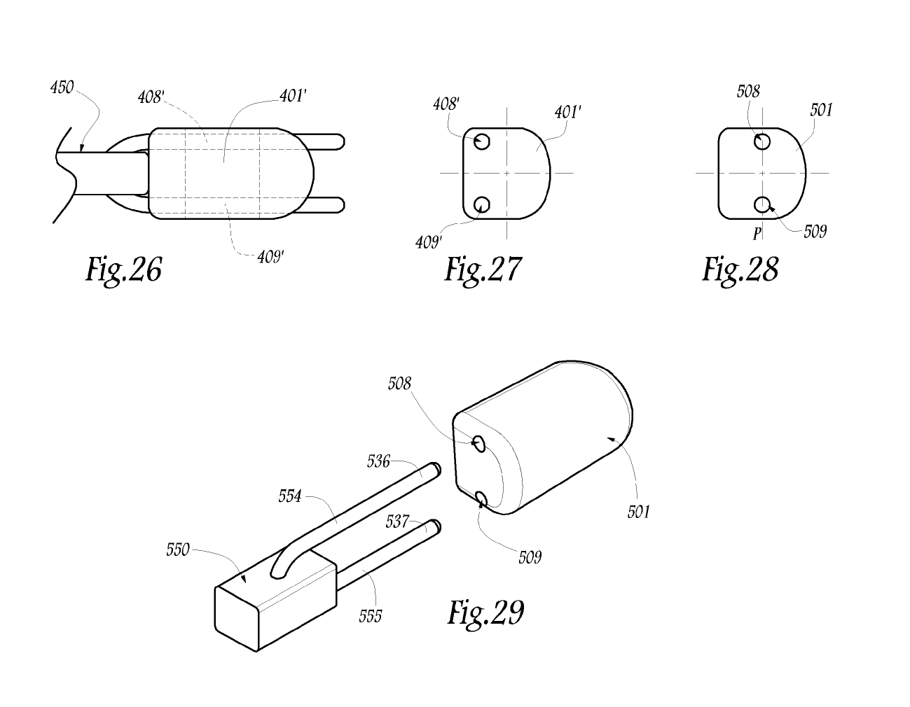

FIGS. 26 and 27 are respectively top and rear views illustrating another alternative form of the invention; and

FIGS. 28 and 29 are respectively rear and perspective views illustrating an embodiment which does not form part of the invention;

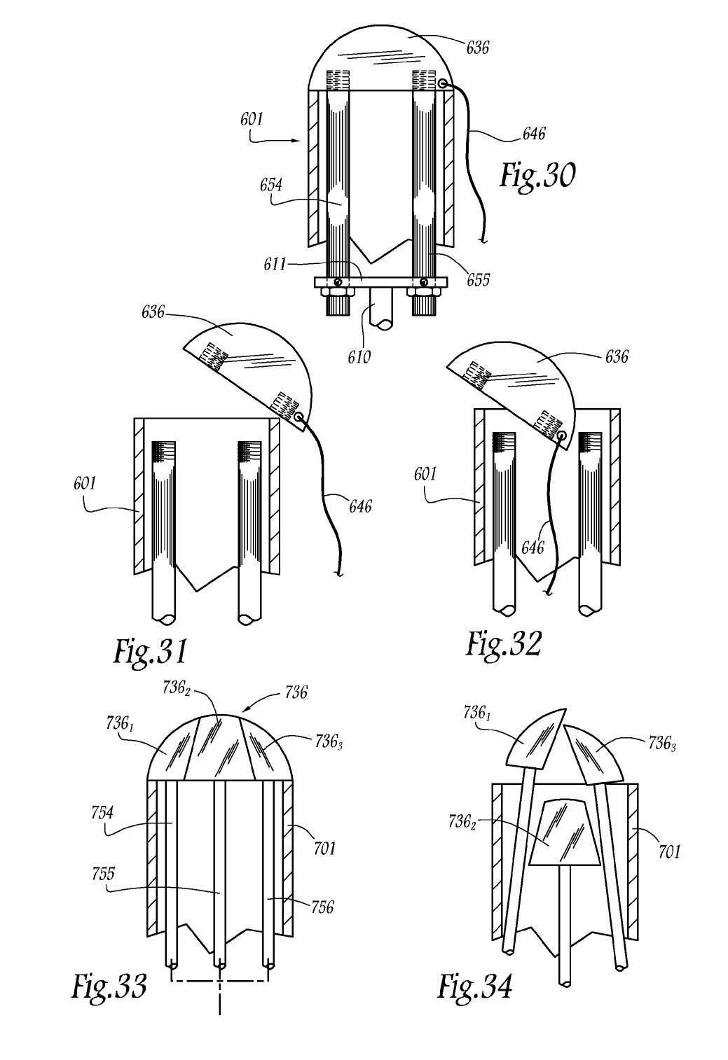

FIGS. 30 to 32 are views in longitudinal section illustrating three steps in the implementation of an additional embodiment of the invention;

FIGS. 33 and 34 are views in longitudinal section illustrating two steps in the implementation of another embodiment of the invention;

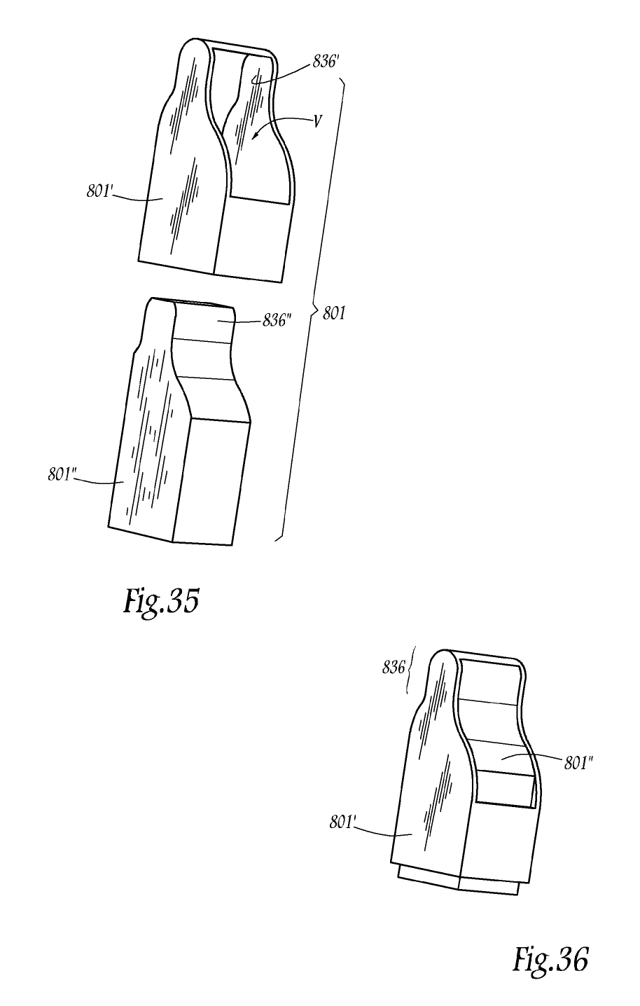

FIGS. 35 to 38 are perspective views illustrating four steps in the implementation of an additional form of the invention;

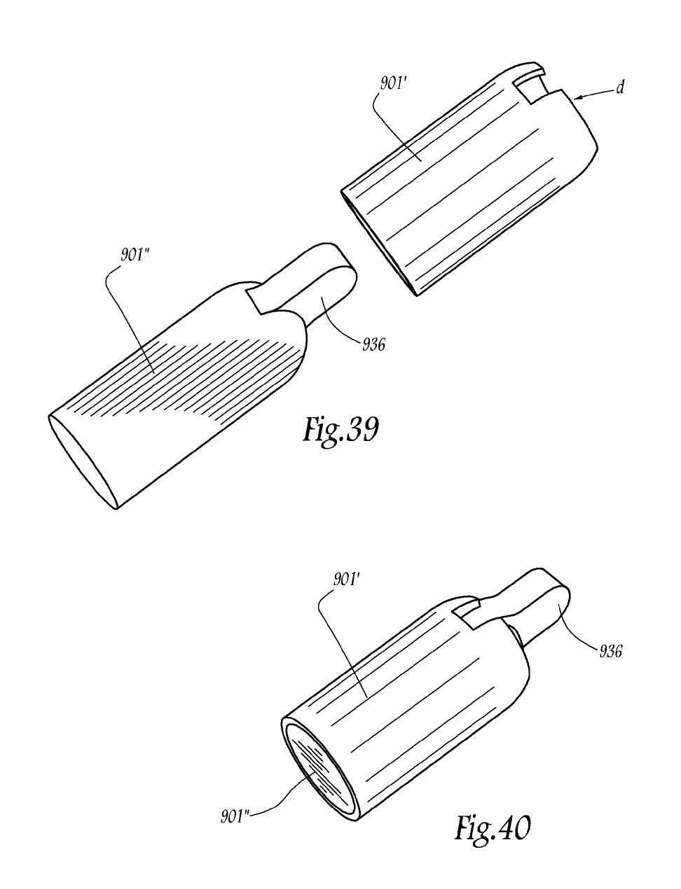

FIGS. 39 and 40 are perspective views illustrating two steps in the implementation of an additional embodiment of the invention;

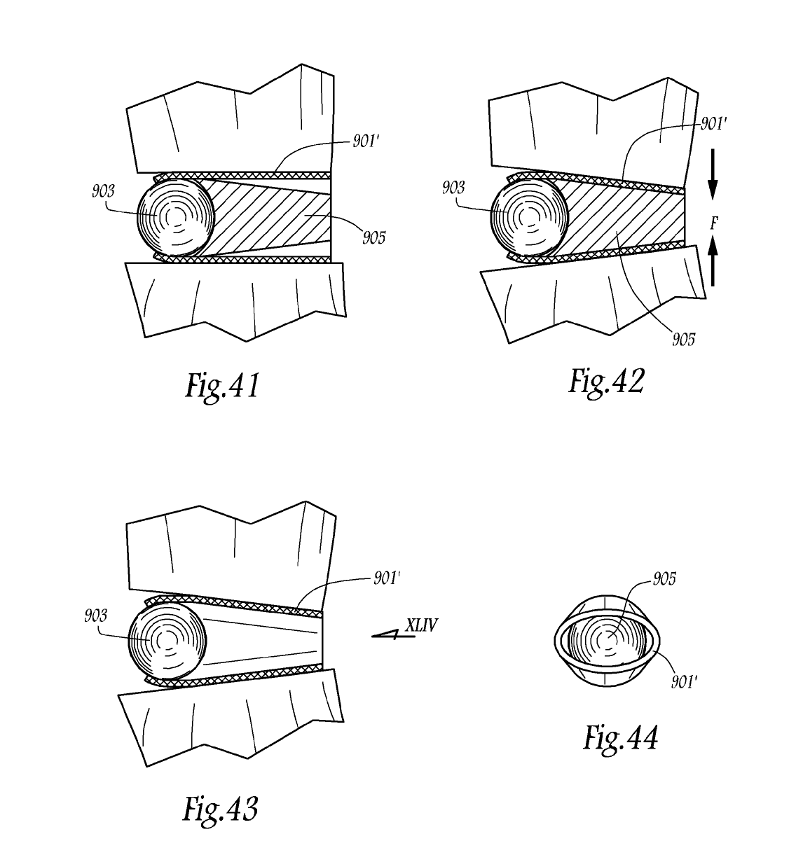

FIGS. 41 and 42 are views in longitudinal section illustrating two additional steps in the implementation of the embodiment of FIGS. 39 and 40;

FIGS. 43 and 44 are respectively views in longitudinal section and end-on of an additional embodiment of the invention;

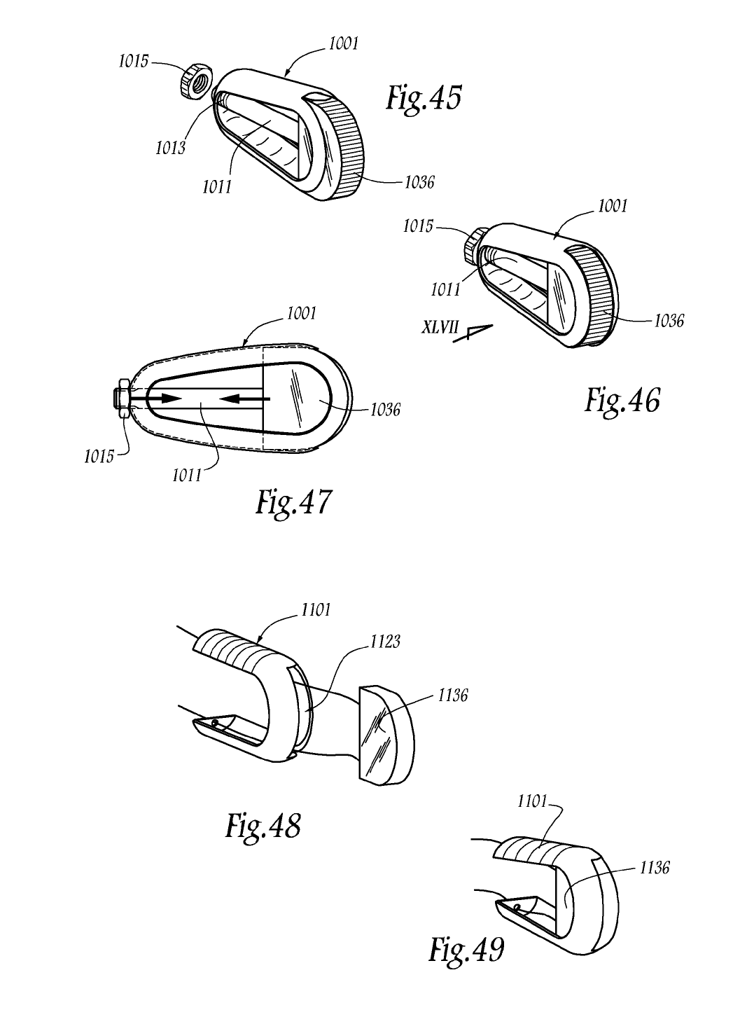

FIGS. 45 to 47 are respectively perspective and front views illustrating yet another embodiment of the invention;

FIGS. 48 and 49 are two perspective views illustrating the implementation of another embodiment of the invention;

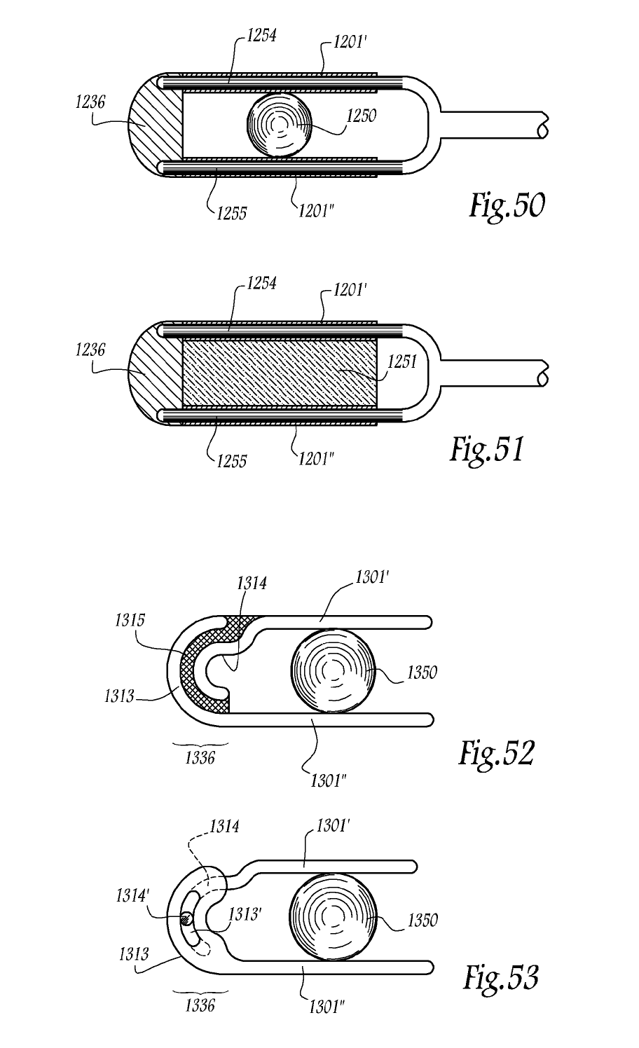

FIGS. 50 to 53 are front views, with part section, illustrating four other embodiments, only the embodiment of FIG. 51 forming part of the invention.

FIGS. 1 to 8 illustrate a first embodiment in which the stabilization assembly according to the invention comprises a cage body, denoted overall by the reference 1, and a removable insert 30 which notably comprises a nose. In that which follows, the terms relating to positioning, notably such as lower, upper, front and rear, relate to a cage body implanted between two vertebrae of a patient, himself in a standing position. Thus, in FIGS. 3, 4 and 5, the cage body is depicted respectively from the side, from the front and from the top.

The cage body 1 represents a substantially parallelepipedal shape. When viewed from the side, it has a height that increases substantially continuously in the forward direction. Thus h and H are used to denote the extreme heights, namely respectively the rear and front heights of this cage body 1 (to the left and right in FIG. 3). L and l are likewise used to denote the length and width of this cage body, both of which are substantially constant.

The longitudinal median axis of the cage body, which extends rear to front, is also denoted A. Finally, α and α′ are used to denote the so-called lordosis-inducing angles, which are formed between the main axis A and the respectively upper 2 and lower 3 surfaces of the cage body.

In terms of numerical values, and purely by way of indication, the height h ranges between 5 and 8 mm, the height H ranges between 7 and 15 mm, the length L ranges between 20 and 25 mm, while the width l ranges between 12 and 14 mm. In addition, the angles α and α′ are advantageously equal, and range in value between 4 and 8°. Under such conditions, the overall lordosis-inducing angle, corresponding to the angle (α+α′), ranges between 8 and 16° and is notably close to 15°.

The cage body 1, of parallelepipedal shape, is truncated by a rounded lateral edge 4 that has no sharp edges and the function of which will be detailed hereinbelow. In addition, the rear face 6 of the body 1 is hollowed with a transverse cut 8 which communicates with an axial opening 10 for the passage of a tool. Finally, at the front face 12, this opening 10 opens into a housing 14 of parallelepipedal shape, the walls of which are square in cross section.

The upper 2 and lower 3 surfaces mentioned hereinabove have a notched surface so as, in the conventional way, to catch more firmly on the vertebral plates. A cutout 18 is also hollowed into the cage body 1, connecting these upper and lower surfaces. This cutout allows the passage of a bone graft, in a way also known per se.

The stabilization assembly according to the invention further comprises the aforementioned insert 30, which comprises a parallelepipedal mount 32, the shape of which complements that of the housing 14. This mount 32 is hollowed by an axial tapping 34 which extends in the continuation of the opening 10.

The mount 32 further supports a nose 36, projecting toward the front of the cage body. When viewed from the side, in FIG. 3, this nose forms the continuation of this cage body, namely its height is equal to the maximum height H of the cage body. By contrast, when viewed from the front and from the top, in FIGS. 4 and 5, this nose is eccentric with respect to the cage body.

More specifically, if the vertical mid-plane of the cage body which contains the axis A is denoted P, the nose 36 is positioned on the opposite side of this mid-plane to the lateral edge 4. The longitudinal axis of the nose, when viewed from above in FIG. 5, is thus denoted A′, and the vertical mid-plane of the nose, when viewed from the front in FIG. 4, is denoted P′. It will be noted that A′ and P′ are offset from the main axis A and the main plane P of the cage body 1, respectively.

The magnitude of the abovementioned offset is denoted d in FIGS. 4 and 5. Advantageously, d ranges between 15 and 30% of the width l of the cage body. Additionally, if the width of the nose is denoted l′, this width l′ is markedly smaller than the width l of the cage body whereas, as was seen earlier, the height of this nose corresponds to the tallest height H of the cage body. Under these conditions, the nose 36 has a small dimension, corresponding to its width, and a large dimension corresponding to its height.

The length of the nose, namely the distance by which this nose projects forward, from the front face of the cage body 1, is also denoted L′. Advantageously, this length L′ ranges between 15 and 25% of the total length L of the cage body. Finally, the nose 36 has a tapered end tip 38, allowing this nose to be inserted more easily between the adjacent vertebrae.

FIG. 6 illustrates an ancillary 50 that can be used to implant the stabilization assembly of the preceding figures, formed of the cage body 1 and of the insert 30. This ancillary 50 comprises a grasping member 52, which supports a rod 54, mounted such that it can slide, and which is able to penetrate the opening 10 of the cage body 1. This rod 54 has a threaded end 56 able to collaborate with the tapping 34 of the insert 30. In addition, on each side of this rod 54, the grasping member 52 is secured to two ribs 58 which can be set into the cut 8 in the cage body 1.

In order to implant the cage body 1 and its insert 30 the intervertebral space illustrated schematically in FIG. 7 is first of all prepared. The two adjacent vertebrae are denoted V₁ and V₂, and the intervertebral space proper is denoted E. This preparation is performed using dilating bougies of conventional type which have not been depicted. In addition, the vertebral plates are abraded, likewise in the usual way.

Moreover, the insert 30 is immobilized with respect to the body 1, while at the same time blocking this body 1 and the ancillary 50 relative to one another. To do this, the mount 32 is positioned in the housing 14 and the threaded end 56 of the rod 54 is screwed into the tapping 34. In addition, the two ribs 58 are locked in the cut 8. Under these conditions, the cage body 1, the insert 30 and the ancillary 50 are secured to one another.

The nose 36 is then introduced into the intervertebral space, via its smallest dimension, namely its width. As FIG. 8 shows, this insertion is performed on one of the sides of the intervertebral space, in this instance the left-hand side, namely some distance from the median axis M of the spinal column. The main root is also denoted R, and the lateral nerve endings are denoted T.

Let P″ be the median horizontal plane of the intervertebral space. Once the nose has been inserted via its smallest dimension, this plane P″ coincides with the main plane P′ of the nose, whereas the main plane P of the cage body 1 is offset from the mid-plane P″. In general, this plane P usually lies below the two planes P′ and P″.

However, if there is only a small offset between the main plane P of the body 1 and the main plane P′ of the nose, it is then possible to position most of the cage body above the mid-plane of the intervertebral space. By contrast, in the event of a larger offset, the option illustrated in FIG. 8 will preferably be adopted, where the most part of the cage body is situated below the intervertebral space, namely facing the lower vertebra V₂. In addition, the main axis A′ of the nose passes through the intervertebral space E, whereas the main axis A of the cage body passes through this lower vertebra.

The surgeon then operates the grasping member 52, turning it in the counterclockwise direction embodied by the arrow F (see FIG. 9). Given that the nose is driven into the intervertebral space, this turning is performed about the main axis A′ of the nose. Now, because the cage body is not symmetric with respect to this main axis, this rotation is accompanied by a cam effect.

In other words, this rotation causes the cage body 1 to move toward the center of the spinal column. In that way, the rounded edge 4 comes into contact with the roots R and endings T, and this tends to push them toward the center of the column, in the direction of the arrow F′. This moving of the nerves is performed gently, given that the lateral edge 4 has no sharp edges and that this movement takes place gradually.

As illustrated in FIG. 9, on completion of the abovementioned rotation, which takes place over approximately ¼ of a turn, the nose 36 penetrates the intervertebral space E via its height H, namely its longest dimension. This intervertebral space therefore finds itself made much taller, so that it becomes far easier for the cage body to be inserted axially.

In FIGS. 8 and 9, the cage body is positioned to the left of the median axis, and it is turned in the counterclockwise direction. If the cage body is situated to the right, then it will of course have to be turned clockwise so that its rounded edge can push against the nerve roots.

Finally, having caused the nose to pivot, this nose now being in its position of FIG. 9, the cage body 1 is driven axially into the intervertebral space E. When the cage body is in place, its respectively upper 2 and lower 3 surfaces press against the plates of the facing vertebrae V₁ and V₂. In addition, the lateral edge 4 faces toward the center of the spinal column.

Once the cage body has been inserted, the rod 54 is unscrewed from the tapping 34 so that the insert 30 supporting the nose 36 is no longer secured to the cage body 1. It then becomes possible for this insert to be pushed back, using a suitable pusher, not depicted. This insert 30 then drops onto the plate of the lower vertebra V₂.

Advantageously, the insert 30 is made of a biocompatible material capable of forming a bone graft. Under such conditions, it is, for example, PEEK or BMP (bone morphogenic protein). Finally, once the insert 30 has been detached from the cage body 1, the ribs 58 are withdrawn from the cut 8, to free the ancillary 50 from this cage body set in place in the intervertebral space.

FIG. 10 illustrates a first alternative form of the invention, which relates more specifically to the way in which the implanting ancillary is secured to the cage body. In this FIG. 10, mechanical elements analogous to those of the first embodiment are associated with the same numerals but with a “prime” suffix.

In contrast to the first embodiment, there is no collaboration here between two ribs and a transverse cut. By contrast, the grasping member 52′ is provided with two fingers 58′ which penetrate blind holes 8′ formed opposite in the cage body 1′.

In order to secure the ancillary 50′ to the cage body 1′ the fingers 58′ are introduced into the blind holes 8′, screwing the threaded end 56′ into the tapping 34′. The cage body 1 is implanted by means of the nose 36′ in a similar way to the way described with reference to the first embodiment. This first alternative form is advantageous insofar as it ensures satisfactory grasp of the cage at the time of turning.

FIG. 11 illustrates a second alternative form of the invention. In this FIG. 11, mechanical elements analogous to those of the first embodiment are assigned the same references, with a “double prime” suffix.

By contrast with the first two embodiments, the nose 36″ is made as a single piece with the cage body 1″. In other words, this body and this nose form a one-piece assembly. As a result, the cage body has no housing, like the housing 14 of the first embodiment. Its front face 12″ is extended directly by the nose 36″ which is formed as an integral part thereof.

Furthermore, the implanting ancillary, not depicted, is analogous to one of these described with reference to the first two embodiments. However, this ancillary has no rod, like the rod 54 in FIG. 6. The ancillary may be provided with a single transverse rib able to penetrate a cut 8″ in the cage body. However, provision may be made for this ancillary to be provided with fingers, like the fingers 58′, able to penetrate two blind holes, like the blind holes 8′ in FIG. 10.

This second alternative form is advantageous, notably in terms of simplicity. Specifically, this cage has a simple mechanical structure accompanied by good robustness. In addition, it entails a low number of handling operations.

According to an additional alternative form, the cage body may be associated with a nose, which is not attached removably as in FIGS. 1 to 10 nor is it of one piece as in FIG. 11. In this alternative form depicted in FIGS. 12 and 13, this nose 236 is retractable, namely is able to occupy two different axial positions.

In the first of these axial positions, the nose projects beyond the cage body 201 so that it can be introduced into the intervertebral space in order to perform the cam effect of the eccentric cage body. This nose is then turned, as explained previously, so as to make the intervertebral space taller, then the cage is inserted axially into this space. Finally, this nose is retracted into the cage body so that it no longer projects toward the front thereof. This is advantageous because this nose then does not occupy any additional space within the intervertebral space.

In the alternative form of FIGS. 12 and 13, the nose is blocked in its axially retracted position and in its axially forward position by any appropriate means, notably by virtue of a rivet. In addition, it will be noted that, in FIGS. 12 and 13, the cage body has a frustoconical rear part and a front part of constant height, in which the nose is housed in its retracted position. However, this retractable nose can be applied to a cage the height of which is constant or alternatively varies continuously along its entire length.

FIG. 14 et seq. illustrate a third alternative form. In this figure, mechanical elements analogous to those of the first embodiment are assigned the same reference numerals, increased by 100.

The cage body 101 differs from that 1 of the first embodiment notably in that it has no transverse cut. The latter is replaced by two longitudinal grooves 108 and 109 hollowed out respectively in the upper 102 and lower 103 surfaces.

When viewed from above, these two grooves extend along an axis A″ which is offset from the main axis A of the cage body. By comparison with FIG. 5, this axis A″ substantially corresponds to that A′ of the nose 36. It will therefore be noticed that these grooves run offset from the median axis and from the mid-plane of the cage body.

In addition, by comparison with the first embodiment, the cage body 101 has no axial opening like the one 10. Nor is there any housing like the housing 14.

The cage body 101 is associated with an implanting member 150, which has a grasping body 152 extended by two rods 154 and 155 capable of penetrating the grooves 108 and 109. When viewed from the side, these two branches extend such that they diverge from the body 152, at the angles α and α′ that correspond to the lordosis-inducing angles of the cage body, the height of which increases toward the front.

Each rod 154, 155 ends in a respective end-piece 136, 137. Unlike these branches, these end-pieces 136 and 137 are “straight”, namely parallel to the main axis A when viewed from the side. Finally, the body 152 is provided with a piston 159 able to move between a retracted position and a position in which it projects forward.

The implanting member 150 and the cage body 101 are secured to one another as follows. First of all, the branches 154 and 155 have to be introduced into the grooves 108 and 109. After this, the end-pieces 136 and 137 project, from the front face 112 of the cage body 101, by a distance L′ substantially corresponding to the length of the nose in the first embodiment.

As FIG. 18 shows, the two end-pieces 136 and 137 are then introduced into the intervertebral space, in a similar way to the way in which the nose 36 is fitted, notably with reference to FIG. 7. At the end of this initial insertion phase, the two end-pieces are positioned side by side, namely the intervertebral space is of small height.

Next, the assembly made up of the cage body 101 and of the implanting member 150 is pivoted in the counterclockwise direction as illustrated in FIG. 19. It will be appreciated that, in this last embodiment, the two end-pieces 136 and 137 perform the same function of eccentric axis of rotation, as the nose 36 of the first embodiment. This being the case, the rounded edge 104 of the cage body pushes back the nerve roots, through a cam effect, as in the first embodiment.

After a rotation through a quarter of a turn, the end-pieces 136 and 137 are now positioned one above the other, so that the intervertebral space E is made significantly taller (FIG. 19). The cage body can then be introduced axially into the intervertebral space as described with reference to the first embodiment. Once this cage body is in position, the piston 159 is actuated and then presses against the rear face 106. This then allows the branches 154 and 155 to be withdrawn from the grooves 108 and 109 so as to detach the implanting member 150 from the cage body 101.

FIGS. 20 and 21 again show a cage body 301 in the shape of a C. This body 301 thus has a web 301₁ intended to be positioned at the front of the intervertebral space, and two flanges 301₂ each of which presses against a respective vertebral body. This cage body 301, which can be likened to a leaf spring, has an open end 301₃ facing toward the rear of the patient, and which can be deformed, preferably when inducing lordosis.

In this example, the deformable cage body 301 is C shaped. However, provision could be made for it to be in accordance with any one of the embodiments described in FR-A-2 913 328 in the name of the present applicant. Thus, the cage body may notably be in the form of a strip defining a closed loop.

Returning to FIGS. 20 and 21, the cage body 301 is associated with a nose 336 which can, for example, be likened to the nose 36″ of FIG. 11. Thus, this nose 336 is eccentric, namely is offset laterally with respect to the longitudinal median axis of the cage body 301. This nose, likewise C-shaped, is located in the continuation of the body 301.

These FIGS. 20 and 21 again show an auxiliary unit 350, forming a cover, made of a substantially rigid material such as PEEK. This unit 350 has a main part 351 that can be inserted between the two flanges of the body 301 and a rounded edge 352 projecting laterally from this cage body.

Thus, for the purposes of implantation, the unit has first of all to be inserted between the two flanges of the cage body, in the position of FIG. 21, so that the edge 352 performs the same function as, for example, the rounded edge 4 illustrated notably in FIG. 5. With a view to implantation, the nose is inserted into the intervertebral space as described notably with reference to FIG. 8, and then this nose and the cage body are pivoted through one quarter of a turn as described notably with reference to FIG. 9.

Once the cage body has been fitted into the intervertebral space, the auxiliary unit is removed using any appropriate means. Under these conditions, this cage body is then able, first of all, to deform so as to allow the vertebrae to be placed in the chosen lordosis position, notably under the action of posterior bracing, then to perform the arthrodesis function as defined hereinabove. In addition, the presence of the unit 350 covers the sharp edges of the C, thus preserving the nerve root.

It will also be noted that the nose, in addition to acting as a cam, performs an additional function of mechanically stabilizing the deformable cage body. Thus, in the example illustrated, this nose prevents the cage body from collapsing near its web.

An additional embodiment according to the invention is illustrated in FIGS. 22 to 25. In these figures, mechanical elements analogous to those of FIGS. 14 to 17 are assigned the same reference numerals increased by 300.

The implantation member 450 differs from that 150 in that it has rods or branches 454 and 455 which end in end-pieces 436 and 437 that extend in the continuation of these rods. In other words, not only the end-pieces but also the rods are parallel to the main axis of the cage body, when viewed from the side.

These rods and these end-pieces enter grooves 408 and 409 formed in the cage body, and which also run parallel to the main axis A, because the branches are not inclined. Finally, the implanting member 450 is provided with a threaded rod 459 able to penetrate a tapped hole 411 formed in the cage body. This allows the cage body to be grasped and, therefore, fitted, more easily.

The various steps in the implanting of the cage body are analogous to those described hereinabove with reference to FIGS. 14 to 17. It will be noted that it is advantageous to provide rods 454 and 455 that are parallel to the main axis A. Indeed, when they are removed from the intervertebral space, these branches are particularly untraumatic to the patient, given that they do not have any roughnesses.

The invention is not restricted to the embodiments described and depicted.

Thus, in most of the embodiments set out hereinabove, the cage body has a height which increases toward the front. However, by way of alternative, an eccentric nose according to the invention can be associated with any type of impaction cage, the height of which is invariable, or even increases in the direction of its rear part.

An embodiment such as this is illustrated in FIGS. 26 and 27 which are to be compared with FIGS. 22 and 25. Indeed, in these various figures, there is one and the same implanting member 450, but the cage bodies differ. In FIGS. 26 and 27, the mechanical elements of this cage body, which are analogous to those of FIGS. 22 to 25, have the same reference numerals but with the “prime” suffix.

As FIG. 26 shows, the cage body 401′ has a height which is unvarying, between its respective front and rear ends. As a result, the grooves 408 and 409 are replaced by two “tunnels”, namely two openings 408′ and 409′ which are formed inside the cage body. In addition, these openings 408′ and 409′, which open both onto the front part and onto the rear part of the cage body, do not, however, open laterally. In this respect, it may be noted that these openings can be created on a pre-existing cage body using any appropriate means such as a drill bit. Such a cage body 401′, of unvarying height, is implanted in the same way as in the previous embodiments.

It will further be noted that, in the various embodiments hereinabove, the nose is eccentric with respect to the cage body, namely that the main axes thereof are separate. However, by way of alternative as depicted in FIGS. 28 and 29, provision may be made for the nose and the cage body to be coaxial. Under these conditions, it is possible to adapt all of the technical features described with reference to all of the figures of the present application to such a coaxial nose. The latter then mainly makes for easy insertion of the cage body into the intervertebral space.

This alternative form is illustrated in FIGS. 28 and 29 which are to be compared with FIGS. 26 and 27. Again there is a cage body 501, pierced with two openings 508 and 509 which are situated in the main plane P, as defined in FIG. 4. These openings are therefore “centered” as opposed notably to those 408′ and 409′ illustrated in FIG. 27.

This cage body 501 collaborates with an implanting member 550 which is provided with two branches 554 and 555 each of which ends in a respective end-piece 536 and 537 which extends in the continuation of this branch. When the aforementioned branches are slipped into the openings 508 and 509, the end-pieces 536 and 537 project in the opposite direction, namely to the right in FIG. 29. These two end-pieces then form a nose of the centered type, allowing the cage body to be inserted into the intervertebral space.

The invention makes it possible to achieve the aforementioned objectives.

Specifically, the cage body according to the invention, aside from performing its conventional intervertebral spacer function, performs an additional function of moving the nerve root aside. This then simplifies the insertion operation, making it less dangerous to the nerve tissue than in the prior art. In addition, by virtue of the invention, it is possible to admit into the intervertebral space cages the height of which is greater than could be admitted in the prior art. Specifically, such tall cages could not be inserted easily using the techniques employed in the prior art.

Finally, the invention makes it possible considerably to simplify the implantation of the intervertebral cage. Specifically, the surgeon is able, in a single action, to perform something that requires several hands in the prior art. This also allows for better control, reduces the number of operations performed “blind” and allows the cage to be fitted more gently.

Various additional alternative forms of the invention will now be described. For each alternative described, the mechanical elements analogous to those of the preceding figures are increased by 100 with respect to the alternative form that immediately precedes it.

FIGS. 30 to 32 first of all show an alternative form in which the branches 654 and 655 do not directly form the nose as they did, for example, in the embodiment of FIG. 22 et seq. These branches are therefore able to collaborate with an insert 636, that forms the insertion nose, and which has smaller dimensions than, for example, the insert 30 of the first embodiment.

The nose 636 may be associated with a wire-like member 646 which notably allows it to be positioned optimally during placement. This wire-like member can also allow the nose to be removed once the cage has been fitted. With a view to insertion, the two branches 654 and 655 are first of all fixed with respect to the nose 636, for example by screwing. It will be noted that, in this embodiment, the branches do not have to extend at the periphery of the cage body 601, namely they may be positioned centrally with respect thereto.

Next, a leg 610 provided with a crosspiece 611 is attached so as to join the two branches together to form a fork. The cage body is then introduced into the intervertebral space as described hereinabove, using the nose 636 and, if appropriate, the wire-like member 646, to achieve better positioning of the nose.

Once the cage body is in place, the two branches are removed from the nose then the nose itself is removed using the wire-like member 646. This removal of the nose can be performed either from the outside of the cage body 601, as in FIG. 31, or from the inside thereof as in FIG. 32.

In addition, as illustrated in FIGS. 33 and 34, provision may be made for the nose 736 to be made of various elements 736₁, 736₂ and 736₃. These elements, which may be assembled with one another at the time of implantation, using any appropriate means, are secured to three branches 754 to 756 of the implanting tool. Once the cage is in place, it is possible to dismantle these three elements, then remove them via the hollow interior of the cage body 701, as shown in FIG. 34.

By way of additional alternative form, the nose 736 may be formed of a number of elements other than three. In addition, the geometric shape of these elements may be other than that described in this figure.

FIG. 35 et seq. illustrate an advantageous alternative form of the invention, in which the cage body 801 is formed of an envelope 801′, and of at least one first insert 801″. More specifically, the or each first insert is intended to collaborate with the envelope, at the time of implantation, whereas an additional insert 803 is provided, the latter being intended to collaborate with the envelope after implantation and after the first insert has been withdrawn from the interior volume of the envelope.

The envelope has a main part ending in a projection 836′ intended to form a nose 836 according to one or other of the preceding embodiments. This envelope defines an interior volume V, being formed, for example, of a thin wall of an appropriate material, notably a biocompatible plastic or metal.

The first insert has the same shape as the envelope and, in particular, a projection 836″ similar to the one 836′. When it has been inserted into its interior volume, this insert 801′ does not project beyond the front of this envelope (FIG. 36). Under these conditions, the assembly formed by the projections 836′ and 836″ of the envelope and of the first insert define an insertion nose 836, within the meaning of the invention, as shown by FIG. 36. This envelope and this first insert are then introduced into the intervertebral space using one of the methods described hereinabove.

Once this insertion has been performed, the first insert needs to be removed from the interior volume of the envelope, leaving this envelope between the vertebrae. It will be noted that the material of which this envelope is made needs to be strong enough that it does not become crushed during the operation, once the first insert has been removed. It will also be noted that this strength does not need to be extremely high because the load exerted by the vertebrae of the anesthetized patient is relatively low. In addition, as will be seen later on, it is advantageous for the envelope to be able to be crushed if a greater force is applied.

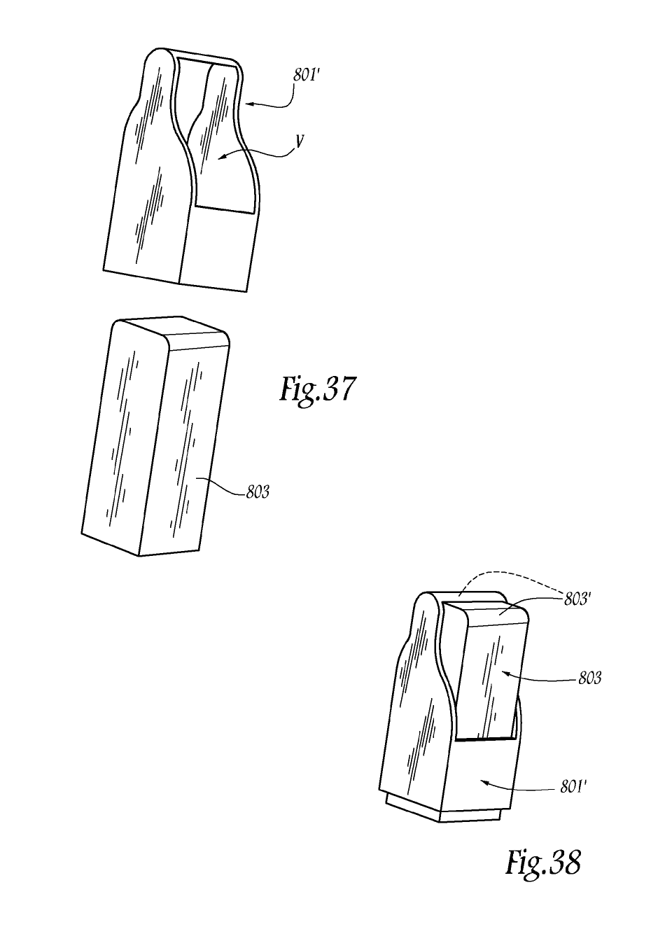

Next, as illustrated in FIGS. 37 and 38, the additional insert 803 is introduced into the interior volume of the envelope 801′. In this regard, it will be noted that this second insert has larger dimensions than this envelope and, in particular, that it projects from the envelope in the form of two lateral bulges 803′. It will be noted that the assembly formed by this second insert and the envelope is no longer asymmetric in shape with a nose, but symmetrical and of parallelepipedal type. This embodiment is advantageous inasmuch as it, on the one hand, allows for easy insertion of the cage body using the “extemporaneous” use of a nose and, on the other hand, allows the use of a customary cage shape if the first insert provided with the nose is replaced by the second insert of solid form.

FIG. 39 et seq. illustrate an additional alternative form, involving a first insert 901″ intended for implantation and several additional inserts 903 and 905 intended to replace this first insert following placement.

By contrast with the previous embodiment, the envelope 901′ is substantially cylindrical, with an anterior part in the form of a section of a sphere, so as to form an opening d of a diameter smaller than that of the cylinder. Moreover, the implanting insert 901″ first of all comprises a main part, of the same shape as the walls of the envelope, which ends in an end-piece 936 forming an eccentric nose within the meaning of the invention (see FIGS. 39 and 40).

The assembly formed by this envelope and this first insert can then be positioned between the vertebrae according to one of the methods described hereinabove. Next, this first insert 901″ is removed and replaced, in this instance, by two additional inserts 903 and 905 (see FIG. 41). More specifically, a sphere 903 is first of all inserted and made to emerge at the anterior part of the envelope. This sphere thus prevents anterior collapse of the envelope. In addition, a third insert 905, the height of which in this instance decreases toward the rear, can be introduced into the posterior part of the envelope.

Once these two additional inserts have been positioned in the interior volume of the envelope, a posterior force F can be applied tending to move the two vertebrae closer together (FIG. 42). This force may be initiated by the patient himself, once he has come round, or alternatively may be initiated by the fitting of a posterior brace. Under these conditions, this brace has a tendency to press the posterior walls of the envelope firmly against the facing walls of the third insert 905 which, as has been seen, taper toward the rear.

FIGS. 43 and 44 illustrate an alternative embodiment to that of the previous figures. Once again there is the anterior sphere 903 which is substantially rigid and performs an anti-collapse function but, on the other hand, there is no third insert. The envelope 901 is likewise deformable, according to the rear force that can be applied by a brace or by the patient himself.

FIGS. 45 to 47 illustrate an additional alternative form of the invention, in which the cage body comprises an envelope 1001 in the form of a tape, as described for example in FR-A-2 913 328 in the name of the present applicant. This tape is associated with a rod 1011 which passes through it from the rear to the front, and which is provided with a screw thread 1013 collaborating with a nut 1015 in the rear part of this tape. In addition, this rod is extended by an end-piece 1036 capable of forming the insertion nose within the meaning of the invention. More specifically, the anterior part of the tape is hollowed with a cutout, through which this end-piece can slide.

In the insertion position illustrated in FIG. 45, the end-piece 1036 projects extensively beyond the cutout, so as to perform its function of insertion nose. Then, once this insertion has been achieved, the surgeon manipulates the threaded rod in such a way as to retract the end-piece into the anterior part of the tape, as illustrated in FIGS. 46 and 47. The surgeon then adjusts the nut in order to place the longitudinal rod in tension. This embodiment is particularly advantageous because, in the first position of FIG. 45, the end-piece acts as a nose and, in the retracted position of FIGS. 46 and 47, it performs an anti-collapse function, while the rod forms a longitudinal brace.

FIGS. 48 and 49 illustrate an additional alternative form of the invention. In this alternative form, there is again an envelope 1101 in the form of a C, as described for example in FR-A-2 913 328 in the name of the present applicant. This C-shaped envelope is hollowed, in its anterior part, by a cutout 1123 that allows the passage of a nose. More specifically, this nose is formed of an end-piece 1136 that can be attached against the walls of the envelope with a view to the introduction of the latter between the vertebrae.

This end-piece may project through the cutout, so as to form the insertion nose. Then, once insertion has been performed in the conventional way, this end-piece can be retracted so as to prevent the envelope from collapsing, as in the previous embodiment (FIG. 49). This end-piece may advantageously be made to move by means of wires 1146 and the use of a rod (not depicted) may also be provided for longitudinal tensioning, as before.

The C-shaped envelope of FIGS. 48 and 49 can be associated with a cover used during implantation, like the one 350 in FIGS. 20 and 21, in which to contain the sharp edges of the C so as to preserve the nerve root. This cover, which collaborates with the cage body during insertion, can also advantageously incorporate the insertion nose 1136. However, two separate members may also perform these respective functions, of protective cover and of insertion nose.

FIGS. 50 and 51 illustrate two additional embodiments. Again there is a cage body formed of two rigid plates 1201′ and 1201″ intended to rest against two corresponding vertebrae, and an interposed body. The latter, which allows mutual displacement between the two rigid plates, may be a sphere 1250 (FIG. 50) or a solid block 1251 (FIG. 51).

Use is made of an insertion fork, the branches 1254 and 1255 of which run longitudinally across the two plates. In addition, a nose 1236 collaborates with these forks, as for example in the embodiment of FIGS. 30 to 32. It will be noted that, when these forks penetrate the plates, this not only allows the nose to be set in place but also allows the plates to be held relative to one another with a view to ease of insertion between the vertebrae. Once the cage body is in place between these vertebrae, the branches of the fork are then withdrawn, together possibly with the nose as described by one or other of the foregoing embodiments.

FIGS. 52 and 53 illustrate another embodiment in which the two plates 1301′ and 1301″ collaborate with one another to form the insertion nose 1336. More specifically, the lower plate has a branch 1313 curved into an arc of a circle, which extends toward the upper plate, while the latter has a hook-shaped projection 1314 extending near this arc-shaped branch. Under these conditions, the nose is formed by this branch and this hook, which extend over a small width of the front part of the plates, when viewed from the front, also being axially offset so as to form an eccentric nose.

In addition, in FIG. 52, an elastomeric material 1315 is advantageously interposed between the branch and the hook, it being understood that this is, however, optional. In this embodiment, the nose, aside from performing its conventional insertion function, performs an additional function of guiding the movement. In this regard it will be noted that the branch and the hook act as a limit stop, both in extension and in flexion, for the two plates relative to one another. This makes it possible to form a guide for the coupling of the vertebral movement, while at the same time also affording a possibility of fore/aft translational movement between the two plates.

In FIG. 53, the hook 1314 is provided with a pin 1314′ capable of sliding along a slot 1313′ formed in the branch 1313. Under these conditions, the relative movement of the plate is dictated by the movement of the pin along this slot, rather than being guided as it was in FIG. 52.

Of the arrangements depicted in FIGS. 50 to 53 only that of FIG. 51 forms part of the invention, using a relatively inelastic interposed block. By contrast, the arrangements using a sphere are of the prosthesis type.

In the various embodiments set out hereinabove, cage bodies intended to be introduced into the intervertebral space have been described. These may be associated with at least one extra-discal posterior element connecting two vertebrae, particularly two adjacent vertebrae. An extra-discal element such as this is able to limit, or even prevent, intervertebral flexion and/or extension movements, so as to remain within the area of arthrodesis.

Such extra-discal elements of the posterior type may be produced in any appropriate form. Mention may, for example, be made of the use of plates or even of more refined devices such as those described in any one of the following French patent applications filed in the name of the applicant:
07 05 371 of 24 Jul. 2007,
08 02 508 of 6 May 2008, and
08 55 574 of 14 Aug. 2008.

Advantageously, these extra-discal elements are segmented, namely connect two adjacent vertebrae. Likewise advantageously, they are articulated on the two screws, notably of the pedicle screw type, that they connect. Finally, these extra-discal elements are tensioned so as to form a brace or a stay, it being understood that it is possible to combine two distinct elements respectively forming a brace and a stay.

According to an advantageous alternative form of the invention which has not been depicted, existing cage bodies can be equipped with attached elements intended to form the insertion nose. These attached elements are thus somewhat like caps that are fitted to the anterior face of the conventional cage body. Such a cap can be positioned against the cage body or alternatively can be connected more firmly to this cage body. In the latter option, provision may be made for the cap to have a relatively flexible fixing region able to fit over the cage body, and a rigid end region forming the nose.

In the various embodiments described hereinabove as forming part of the invention, use is made of mechanical components suited to performing arthrodesis as defined at the beginning of the present description. The person skilled in the art will therefore assign the appropriate features to these components, particularly in terms of their stiffness, while at the same time taking into consideration the way in which they are combined with any posterior element that might be used.

1-36. (canceled)

37. An intervertebral stabilization assembly for arthrodesis, comprising a cage body, intended to be introduced by impaction into the intervertebral space, this assembly further comprising a nose for introducing the cage body into the intervertebral space, this nose being secured to or able to be secured to this cage body, in an eccentric position, so that when this nose is introduced into the intervertebral space, the rotating of this nose causes this intervertebral space to open up and, at the same time, causes the cage body to move in the manner of a cam.

38. The assembly as claimed in claim 37, wherein, when viewed from above, the cage body and the nose have respective main axes which are separate.

39. The assembly as claimed in claim 38, wherein the main axes of the nose and of the cage body are parallel.

40. The assembly as claimed in claim 37, wherein the cage body has a rounded exterior edge, on the opposite side from the nose with respect to a mid-plane of this body.

41. The assembly as claimed in claim 37, wherein, when viewed from the side, the dimensions of the cage body decrease toward the rear.

42. The assembly as claimed in claim 41, wherein the exterior walls of the cage body, which are intended to come into contact with the vertebral bodies, define a lordosis-inducing angle greater than 10°, notably close to 15°.

43. The assembly as claimed in claim 37, wherein, in at least one insertion position, the nose projects forward, from a front face of the cage body.

44. The assembly as claimed in claim 37, wherein, when viewed from the side, the nose has the same height as the tallest part of the cage body.

45. The assembly as claimed in claim 37, wherein the cage body comprises an envelope defining an interior volume, and wherein a first insert, or implanting insert, is provided, this insert being able to be attached inside the interior volume of the envelope so as to form the nose.

46. The stabilization assembly as claimed in claim 45, wherein the implanting insert can be removed from the interior volume of the envelope, and wherein at least one additional insert is provided, the latter being able to be introduced into the interior volume of the envelope in place of the implanting insert.

47. The assembly as claimed in claim 45, wherein the envelope can be deformed, notably with a view to adopting a lordosed configuration.

48. The assembly as claimed in claim 37, wherein the nose is able to adopt at least one position protruding in relation to the cage body, in which position it allows this cage body to be inserted, and a position retracted inside the cage body, in which position it performs an anterior anti-collapse function.

49. The assembly as claimed in claim 48, wherein the retractable nose is associated with an adjusting rod which performs a longitudinal bracing function when the nose is in the retracted position.

50. The assembly as claimed in claim 37, wherein the nose is produced as one piece with the cage body.

51. The assembly as claimed in claim 37, wherein the nose is formed in an insert able to be attached removably in relation to the cage body.

52. The assembly as claimed in claim 51, wherein the cage body has hollowed into it a housing in which there is housed a mount from which the nose extends.

53. The assembly as claimed in claim 52, wherein the walls of the housing and of the mount are such that they prevent any mutual rotation, notably having a polygonal, notably square, shape.

54. The assembly as claimed in claim 52, wherein the mount is tapped.

55. The assembly as claimed in claim 51, wherein the nose can be fixed, removably, to at least two branches of an implanting tool.

56. The assembly as claimed in claim 55, wherein the nose is formed of several elements, these various elements being able to be assembled with one another at the time of insertion, while these various elements can be detached from one another after insertion, so as to be removed, notably via the interior volume of the cage body.

57. The assembly as claimed in claim 51, wherein the nose is made of a material that allows bone grafting.

58. The assembly as claimed in claim 37, wherein the stabilization assembly further comprises an implanting member for implanting the cage body, and the nose is formed of part of this implanting member, in a position in which this member and this cage body are secured to one another.

59. The assembly as claimed in claim 58, wherein the cage body has, hollowed into it, eccentric passage means while the implanting member comprises two branches able to penetrate these passage means, these branches ending in two end-pieces able to project toward the front of these passage means, so as to form the nose.

60. The assembly as claimed in claim 59, wherein each end-piece extends in the continuation of a respective branch.

61. The assembly as claimed in claim 59, wherein the implanting member further comprises a piston able to push the cage body (101) away from the branches.

62. The assembly as claimed in claim 59, wherein the implanting member comprises a threaded rod able to penetrate a tapped hole formed in the cage body.

63. The assembly as claimed in claim 59, wherein the passage means are grooves hollowed into the cage body.

64. The assembly as claimed in claim 59, wherein the passage means are eccentric openings opening onto the front and rear faces of the cage body but not opening onto the lateral faces of the cage body.

65. The assembly as claimed in claim 37, further comprising at least one posterior extra-discal element able to limit, or even prevent, intervertebral flexion and/or extension.

66. The assembly as claimed in claim 37, wherein a cover is provided to cover the sharp edges of the cage body during insertion.

67. The assembly as claimed in claim 66, wherein the cover is provided with the insertion nose.

68. The stabilization assembly as claimed in claim 37, wherein the cage body comprises two plates intended to rest against the vertebrae, and an intermediate element able to provide relative travel between these plates, the nose being secured to an implanting tool comprising two branches extending into these two plates.

69. An ancillary for implanting the stabilization assembly as claimed in claim 50, comprising a grasping member for a surgeon to grasp, blocking means for blocking the cage body with respect to the grasping member, and immobilizing means for immobilizing the nose with respect to the cage body.

70. The ancillary as claimed in claim 69, wherein the blocking means comprise at least one rib of the grasping member, able to collaborate with a groove formed by the cage body.

71. The ancillary as claimed in claim 69, wherein the blocking means comprise at least two fingers of the grasping member which are able to enter at least two orifices formed in the cage body.

72. The ancillary as claimed in claim 69, wherein the immobilizing means comprise a threaded rod of the grasping member able to penetrate a tapped orifice of the insert provided with the nose.
